(19) United States
(12) Patent Application Publication
(10) Pub. No.: US 2015/0101615 A1
(43) Pub. Date: Apr. 16, 2015

(54) Systems, Devices, and Methods for Retaining Oral Devices for Airway Treatment

(71) Applicant: APNICURE, INC., Redwood City, CA (US)

(72) Inventors: Jonathan L. PODMORE, San Carlos, CA (US); D. Heath ROBERTS, Allen, TX (US)

(21) Appl. No.: 14/511,404

(22) Filed: Oct. 10, 2014

Related U.S. Application Data
(60) Provisional application No. 61/889,584, filed on Oct. 11, 2013.

(51) Int. Cl. A61F 5/56

(52) U.S. Cl. CPC A61F 5/566
USPC 128/848

(57) ABSTRACT

Systems, devices, and methods for retaining a treatment device in the patient's oral cavity through the course of treatment are provided. Retainer elements, which match the upper and lower teeth of the patient, are worn by the patient and coupled to the bite plate of the treatment device. The upper and lower retainer elements can be coupled to the bite plate or otherwise positioned relative to one another such that the lower teeth and mandible are advanced in the anterior direction, which helps maintain airway patency. The treatment device can also apply a vacuum to the oral cavity to help maintain airway patency.

### CROSS-REFERENCE

This application claims the benefit of U.S. Provisional Application No. 61/889,584 (Attorney Docket No. 41506-714.101), filed Oct. 11, 2013, which application is incorporated herein by reference.

### BACKGROUND OF THE INVENTION

The present invention relates generally to medical devices and methods. In particular, the present invention relates to systems, devices, and methods for retaining oral devices for airway treatment in the oral cavity of the patient.

Obstructive sleep apnea (OSA) is a serious medical condition resulting from a temporary airway blockage which occurs as a patient sleeps. The airway blockage usually occurs between the soft palate and/or the back of the tongue and the pharynx. As the patient breathes, the reduced area in the upper airway can cause snoring, and more seriously, OSA.

Sleep disruption caused by OSA can result in severe daytime sleepiness, chronic fatigue, headaches, depression, accidents, injuries, and of particular concern, OSA can reduce the amount of oxygen entering the lungs causing hypoxia. Hypoxia, in turn, can lead to pulmonary hypertension, heart disease, and stroke.

Numerous invasive and less invasive treatments have been proposed for OSA. Of particular interest to the present disclosure, “continuous positive airway pressure” (CPAP) delivers a continuous stream of pressurized air directly to the person's upper airway. The positive pressure maintains patency of the airway and inhibits the collapse associated with OSA. Although generally effective, CPAP suffers from a number of drawbacks that have led to a high level of non-compliance. The patient must wear a bulky facial mask which can be uncomfortable, and the system generates noise that can make falling asleep difficult. CPAP is also difficult to use because the mask requires careful fitting to avoid air leaks and facial discomfort and because the mask can easily be dislodged during sleep. Moreover, a number of unpleasant side effects, such as sore throats, dry throat and eyes, headaches, and skin rashes from the mask frequently occur. These problems have resulted in a high level of non-compliance with CPAP therapy.

As an improvement over CPAP, it has been proposed to apply a negative pressure to the patient's oral cavity. For example, devices have been proposed which apply a vacuum at the forward end of the patient's mouth, typically at or just behind the lips, to pull the tongue forward in order to lift the rear portion of the tongue away from the back of the airway. See, for example, U.S. Patent Publication Nos. 2007/0277818, 2005/0166928 and 2005/0166929. As an improvement over these devices, it has more recently been proposed to apply a negative pressure in region or space above the tongue which in turn draws the soft palate away from the pharynx to draw the rear portion of the tongue away from the pharynx as well. See, commonly owned U.S. Patent Publication Nos. 2009/0120446 and 2009/0120447.

As another alternative to CPAP, the use of mandibular advancement devices, often referred to as a mandibular splint or a mandibular advancement splint, has been proposed. A mandibular advancement device is an oral appliance worn in the mouth over the upper and lower teeth. The device treats snoring and sleep apnea by moving the lower jaw forward slightly, which tightens the soft tissue and muscles of the upper airway to inhibit obstruction of the airway during sleep. The tightening created by the device may also suppress vibrating of the soft tissues of the upper airway as air passes over them, which is the most common cause of loud snoring. Mandibular advancement can be used in combination with negative pressure therapy to clear the airway. See, commonly owned U.S. Patent Publication No. 2013/0186411. Other mandibular advancement devices are described in U.S. Patent Publication Nos. 2005/0028826, 2007/0074729, 2010/0043805, and 2011/000495. While often effective, such mandibular advancement devices can cause significant jaw discomfort and can cause teeth to shift over time, leading to significant dental problems.

Another recent significant improvement in the treatment of sleep apnea is described in U.S. Pat. Nos. 8,074,656; 8,074,656; and 8,122,889, U.S. Patent Publication No. 2012/0017917, and co-pending application Ser. No. 13/593,137, each assigned to the assignee of the present application, the full disclosures of which are incorporated herein by reference. These patents and applications describe engaging a lateral element across a medial region of the tongue to create a clearance above the tongue. By drawing a vacuum in the oral cavity, the soft palate can be drawn forward to open the airway. While very successful in many patients, the treatment is not fully effective in some patients.

With all such devices held in the oral cavity, retention of the device can be a challenge. These devices are often held in the patient's oral cavity for hours at a time as the patient is sleeping. Often times, a patient will move in his or her sleep and such movement may cause the oral device to loosen or even fall entirely out of the oral cavity. In at least some instances, a patient may unconsciously attempt to speak during sleep which can also pose a challenge for device retention. Retention of the treatment devices in the oral cavity can therefore be crucial for implementing the negative pressure, mandibular advancement, or vacuum therapy through a desired time period.

For these reasons, it would be desirable to provide alternative and improved methods and apparatus for treating obstructive sleep apnea and snoring. The methods and devices should be non-invasive and require no surgery or permanently implanted components. In addition, the methods and devices should be minimally intrusive with components that are comfortable and quiet so that disruption of the patient's sleep as well as the risks of the device being displaced or falling out entirely are minimized. The methods and devices should also reduce any risk of tooth displacement associated with mandibular advancement or other causes. Moreover, the methods and devices should avoid contacting the portions of the oral cavity that trigger the gag reflex. The methods and systems should also be simple to implement and be effective to significantly improve patency of a patient's airway during sleep in a broad population of patients suffering from sleep apnea. At least some of these objectives will be met by many embodiments described hereinafter.

### SUMMARY OF THE INVENTION

The present disclosure provides systems, devices, and methods for retaining a treatment device in the patient's oral cavity through the course of treatment. Such treatment devices will typically be for maintaining patency (openness) in a patient's airway. The devices will typically be configured for at least one of mandibular advancement or applying a vacuum to the oral cavity. Such treatment devices are often in the form of a bite plate held between the upper and lower teeth of the patient. Retainer elements, which match the at least one of the upper or lower teeth of the patient, can be worn by the patient and can be coupled to the bite plate to improve retention of the bite structure. The upper and lower retainer elements can also be coupled to the bite structure or to each other so that their positioning relative to one another can cause mandibular advancement.

An aspect of the disclosure provides an oral device for temporary placement in a patient's oral cavity. The device typically comprises a base, a lip seal, and at least one port. The base can be adapted to be held between the patient's upper and lower teeth and is often shaped to accommodate the patient's tongue when held therebetween. The lip seal can retain the oral device in the patient's oral cavity and is often adapted to be held between the inner surface of the patient's lips and the anterior surface of the patient's teeth. The port(s) may be adapted to be connected to a vacuum source to apply a partial vacuum in the patient's oral cavity. The applied vacuum can draw together the soft palate and the posterior region of the tongue to maintain airway patency. Alternatively or in combination, the applied vacuum can advance the tongue forward (in the anterior direction) to maintain airway patency. The base, the lip seal, or both can be adapted to couple to at least one retainer element configured to couple and conform to the outer surfaces of the patient's upper or lower teeth.

The oral device may further comprise a tongue-engaging member coupled to the base. The tongue-engaging member may be disposed to engage a medial region of the tongue when the base is positioned between the patient's teeth.

The port(s) may be positioned on at least one of the tongue-engaging member or the base. The port(s) may be adapted to be connected to a vacuum source. The vacuum source may be separately connected to and adjustable for each of the port(s) positioned on the tongue-engaging member and the port(s) positioned on the base.

The tongue-engaging member may have an anterior surface and a posterior surface. The anterior surface may be inclined forwardly relative to a plane of the base and may be disposed to engage a medial region of the tongue when the base is held between the patient's upper and lower teeth. The tongue-engaging member may have an integral plenum connectable to the vacuum source with at least one port disposed on a posterior surface of the tongue-engaging member and open to the integral plenum. The tongue-engaging member may have a plurality of ports distributed over the posterior surface thereof. The anterior surface and the posterior surface of the tongue-engaging member may be inclined forwardly at an angle in the range from 45 degrees to 60 degrees relative to a plane of the base when positioned between the upper and lower teeth. The anterior surface of the tongue-engaging member may be generally parallel to the posterior surface of the tongue-engaging member.

The oral device may further comprise a tongue pocket coupled to the base. The tongue pocket may have an interior disposed to receive an anterior region of the patient's tongue when the tongue passes through an anterior opening of the base. The tongue pocket may extend in an anterior direction from the lip seal. The port(s) may be positioned on the tongue-engaging member, the base, the tongue pocket, or any combination thereof. The vacuum source may be separately connected to and adjustable for each of the port(s) positioned on the tongue-engaging member, the base, and the tongue pocket. The vacuum source may be adapted to draw a vacuum in the range from 35 cmH₂O to 60 cmH₂O in the tongue pocket and a vacuum in the range from 25 cmH₂O to 50 cmH₂O through the tongue-engaging member. The port(s) positioned on the tongue-engaging member, base, and/or tongue pocket may comprise a vacuum port and a bleed port to allow a continuous air flow therethrough while maintaining a partial vacuum.

The oral device may further comprise an attachment element coupled to the base and/or the lip seal for coupling the retainer element(s) thereto. The attachment element may comprise at least one of a screw, a bolt, a latch, a fastener, or an adhesive. Alternatively or in combination, at least one of the base or the lip seal comprises a first attachment feature configured to couple to a second attachment feature of the at least one retainer, often without requiring any intermediate structure or element. For example, at least one of the base or the lip seal may be molded to have a male or female feature (e.g., a shaped cavity) for attachment to a female or male feature, respectively, molded onto a male or female feature (e.g., a hook for placement within the shaped cavity) molded onto the at least one retainer element.

The retainer element(s) may comprise an upper retainer element configured to couple and conform to the outer surfaces of the patient's upper teeth, a lower retainer element configured to couple and conform to the outer surfaces of the patient's lower teeth, or both the upper and lower retainer elements. The lower retainer element may be positioned relative to the upper retainer element on the base such that the patient's lower teeth and mandible are advanced in the anterior direction from their natural position when the patient is wearing the upper and lower retainer elements.

The base may comprise an upper plate for receiving the upper teeth and a lower plate for receiving the lower teeth. The upper and lower plates may diverge in the anterior direction so that the base holds the patient's upper teeth and lower teeth apart to provide the anterior opening therebetween. The upper and lower plates may each comprise a U-shaped bite plate configured to be positioned against full dentition.

The oral device may be a component of a system for temporary placement in a patient's oral cavity, according to another aspect of the disclosure. The system comprises the oral device and a retainer structure configured to couple and conform to the outer surfaces of the at least one of the patient's upper or lower teeth. The retainer structure may comprise an upper retainer element configured to couple and conform to the outer surfaces of the patient's upper teeth, a lower retainer element configured to couple and conform to the outer surfaces of the patient's lower teeth, or both. The system may further comprise a vacuum source connectable to the oral device.

Another aspect of the disclosure provides a method for stabilizing a soft palate in a patient's oral cavity. A bite plate may be placed in the oral cavity to be held between upper and lower teeth of the patient. The bite plate may be coupled to at least one of the upper or lower teeth of the patient, with at least one of an upper or lower retainer element adapted to conform to the outer surfaces of the at least one of the upper or lower teeth, respectively. A vacuum may be applied to the oral cavity through the at least one of bite plate or a structure coupled to the bite plate. The applied vacuum may draw together the soft palate and a posterior region of the tongue, draw the tongue forward in the anterior direction, or both.

The method may further comprise drawing the lower teeth and the mandible of the patient forward. The lower teeth and the mandible may be drawn together by positioning the lower retainer element relative to the upper retainer element on the bite plate such that the patient's lower teeth and mandible are advanced in the anterior direction from their natural position. Then, the bite plate is coupled to the upper and lower teeth of the patient with the upper and lower retainer elements, respectively.

The structure coupled to the bite plate may comprise a tongue-engaging member. The vacuum may be applied to the oral cavity through the port(s) positioned on at least one of the upper surface of the bite plate, a lower surface of the bite plate, the tongue-engaging member, or any combination thereof. The tongue-engaging member may be engaged against a medial region of the tongue to provide a clearance between the medial region and the palate. The vacuum may be applied to the oral cavity though the tongue-engaging member, for example, to draw together the soft palate and the posterior region of the tongue.

The method may further comprise capturing the tongue in a tongue pocket. The vacuum may be applied in the pocket to draw the tongue forward in the anterior direction. The structure coupled to the bite plate may comprise a tongue-engaging member which may be engaged against a medial region of the tongue to provide a clearance between the medial region and the palate. The vacuum may also be applied to the clearance though the tongue-engaging member to draw together the soft palate and the posterior region of the tongue. The anterior surface of the tongue-engaging member may be inclined forwardly relative to a plane of the base when the base is held between the upper and lower teeth. The anterior surface may engage the medial region of the tongue and displace the medial region of the tongue in an anterior direction relative to the soft palate.

The vacuum may be applied to the clearance through port(s) disposed over the posterior surface of the tone-engaging member. The vacuum in the clearance may draw the soft palate against the posterior surface of the tongue-engaging member. The soft palate may remain spaced-apart from the posterior surface of the tongue-engaging member while the vacuum in the clearance is being applied. The vacuum in the clearance and the vacuum in the tongue pocket may be maintained at different levels. The vacuum in the clearance may be less than the vacuum in the tongue pocket. The vacuum in the clearance is in the range from 25 cmH₂O to 50 cmH₂O and the vacuum in the tongue pocket is in the range from 35 cmH₂O to 60 cmH₂O. The vacuum may be applied by continuously drawing a vacuum while simultaneously bleeding in a controlled amount of air to maintain a desired partial vacuum.

Another aspect of the disclosure provides an oral device for temporary placement in a patient's oral cavity. The device may comprise a base and at least one port. The device may or may not further comprise a lip seal. The base may be adapted to be held between the patient's upper and lower teeth and may be shaped to accommodate the patient's tongue when held therebetween. The port(s) may be adapted to be connected to a vacuum source to apply a partial vacuum in the patient's oral cavity. The base may be adapted to couple to at least one retainer element. The retainer element(s) may be configured to couple and conform to the outer surfaces of at least one of the patient's upper or lower teeth. The oral device may further be configured in many ways in accordance to the many embodiments described herein, such as in accordance with the many embodiments of the first aspect of the disclosure discussed above.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a perspective view of a treatment device for a patient's oral cavity and an upper retainer element that can be coupled to the device, according to many embodiments;

FIG. 1B shows a perspective view of the treatment device coupled to the upper retainer element, according to many embodiments;

FIG. 2A shows a perspective view of the treatment device and upper and lower retainer elements that can be coupled to the device, according to many embodiments;

FIG. 2B shows a perspective view of the treatment device coupled to the upper and lower retainer elements, according to many embodiments;

FIG. 2C shows a side view of the treatment device and the upper and lower retainer elements, according to many embodiments;

FIG. 2D shows a top view of the treatment device, according to many embodiments;

FIG. 2E shows a bottom view of the treatment device, according to many embodiments;

FIG. 2F shows a side view of the treatment device coupled with the upper and lower retainer elements which are positioned to match the natural bite of the patient when worn, according to many embodiments;

FIG. 2G shows a side view of the treatment device coupled with the upper and lower retainer elements which are positioned to advance the lower teeth and mandible of the patient when worn, according to many embodiments;

FIG. 2H shows a side view of the treatment device coupled with the upper and lower retainer elements which are positioned to retract the lower teeth and mandible of the patient when worn, according to many embodiments;

FIG. 3 shows a perspective view of upper and lower retainer elements coupled together to so the lower teeth and the mandible are advanced when worn by the patient, according to many embodiments, according to many embodiments;

FIG. 4A shows a perspective view of the top of treatment device and the separated upper retainer element, according to many embodiments;

FIG. 4B shows a perspective view of the side of treatment device and the upper retainer element coupled together, according to many embodiments;

FIGS. 4C and 4D show perspective views of the top of treatment device and the upper retainer element coupled together, according to many embodiments;

FIG. 4E shows a perspective view of the treatment device, according to many embodiments;

FIG. 4F shows a magnified view of the attachment elements coupling the treatment device and the upper retainer element together, according to many embodiments;

FIG. 4G shows a top view of the upper retainer element coupled to the attachment elements, according to many embodiments; and

FIG. 4H shows a perspective view of the top of the upper retainer element coupled to the attachment elements, according to many embodiments.

### DETAILED DESCRIPTION OF THE INVENTION

Systems, devices, and methods for retaining a treatment device in the patient's oral cavity through the course of treatment are provided. Treatment devices according to many embodiments will be configured for maintaining patency (openness) in a patient's airway by at least one of mandibular advancement or application of a vacuum or negative pressure to the oral cavity. Such treatment devices are often in the form of a bite plate held between the upper and lower teeth of the patient. Retainer elements, which match the upper and lower teeth of the patient, can be worn by the patient and can be coupled to the bite plate to improve retention of the bite structure. The upper and lower retainer elements can also be coupled to at least one of the bite structure or to teach other so that their positioning relative to one another can cause mandibular advancement.

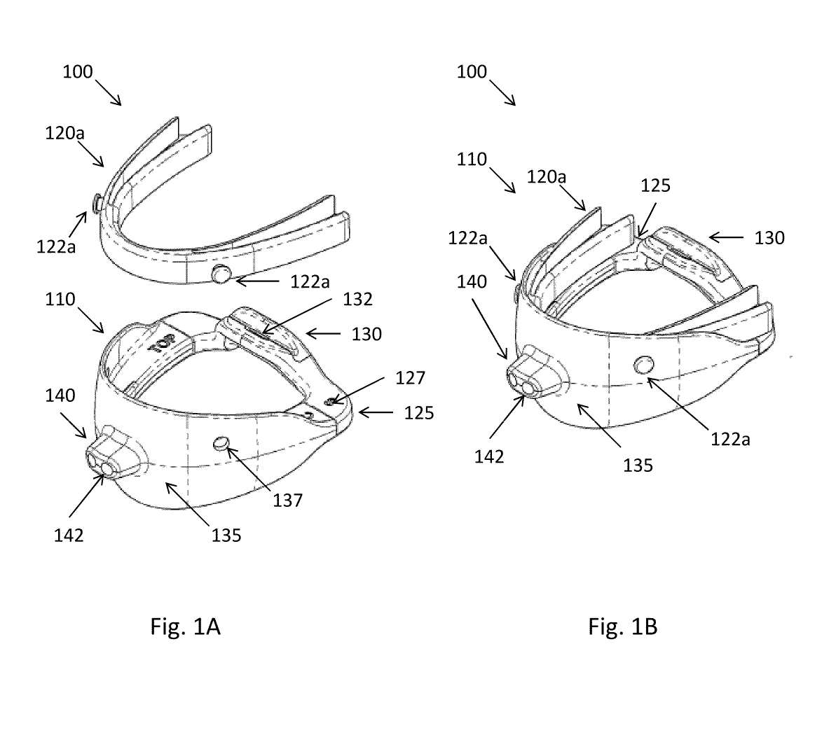

FIG. 1A shows system 100 for the treatment for maintaining airway patency. The treatment system 100 may comprise a treatment device 110 and a superior or an upper retainer element 120a. The treatment device 110 may comprise any one of the airway patency devices described in co-owned U.S. Pat. Nos. 8,074,656, 8,122,889, 8,122,890, 8,367,620, and 8,505,504 and U.S. Patent Publication Nos. 2012/0132215, 2012/0132216, 2012/017917, 2013/0186411, and 2013/0213409. The treatment device 110 alone may be placed in the oral cavity of a patient to engage the medial region of the tongue and/or to apply a negative pressure or vacuum to the oral cavity to maintain airway patency. Retainer elements which couple and conform to the outer surfaces of at least one of the upper or lower teeth can also be coupled to the patient's teeth and the treatment device 110 to improve the retention of the device 110 in the oral cavity when the treatment device 110 is worn. As shown in FIG. 1B, the upper retainer element 120a, which has an interior shape molded to match the outer surfaces of the patient's upper teeth, can be attached to a superior or an upper surface of the treatment device 110. The upper retainer element 120a may be made of a material such as polycaprolactone or ethylene-vinyl acetate.

The treatment device 110 may comprise a U-shaped base 125 and a lip seal 135. The treatment device 110 may also comprise at least one of a tongue-engagement element 130 or a tongue pocket 140. The posterior end of the U-shaped base 125 can be coupled to the tongue-engagement element 130. The anterior face of the U-shaped base 125 can be coupled to the lip seal 135. The anterior ends of the U-shaped base 135 and the lip seal 135 can be open to the tongue pocket 140.

The U-shaped base 135 may have at least one port 127 for applying a vacuum or negative pressure to the patient's oral cavity, which can draw soft palate away from the pharynx to draw the rear portion of the tongue away from the pharynx as well. These port(s) 127 may be disposed on at least one of the upper (superior) or lower (inferior) surface of the U-shaped base 135. The tongue-engagement element 130 may have at least one port 132 for applying a vacuum or negative pressure to the patient's oral cavity. Typically, these ports 132 are oriented superiorly to face the soft palate such that an applied vacuum or negative pressure is applied to the region of the oral cavity between the medial region of tongue and the soft palate. The tongue pocket 140 may have at least one port 142 for apply a vacuum or negative pressure to the anterior tip of the patient's tongue to draw the tongue forward.

The upper retainer element 120a may comprise at least one attachment element 122a and the lip seal 135 may have at least one opening 137 to facilitate the attachment of the upper retainer element 120a to the treatment device 110. For example, the attachment element(s) 122a may comprise button(s) which are pressed through opening(s) 137 to attach the upper retainer element 120a to the upper (superior) portion of the lip seal 135. Alternatively or in combination, the attachment element(s) 122a may attach the upper retainer element 120a to the upper (superior) surface 125a of the U-shaped base 125. The attachment element(s) or button(s) 122a may comprise integral features of the lip seal 135 which are molded thereon. Alternatively or in combination, the attachment element(s) or buttons 122a may comprise separate structures or elements attached to the lip seal 135 such as one or more of a screw, a bracket, a latch, a non-toxic adhesive, or other mechanical or chemical features for attachment or adhesion.

To manufacture the upper or lower retainer element 120a, 120b, a patient may heat and mold the retainer element 120a, 120b in place. The cooled retainer element 120a or 120b would fit the teeth snugly while providing the attachment features or elements. For example, the retainer element 120a or 120b would comprise a rigid portion (for example, made from polycarbonate or ABS), which coupled comprise the attachment features, over which the boil and bite material is over-molded so as to create a dual material tray. Alternatively or in combination, a full boil and bite material tray having a support frame used during the forming could be provided such that the attachment or elements would remain in fixed locations until the retainer element 120a or 120b is set and cooled.

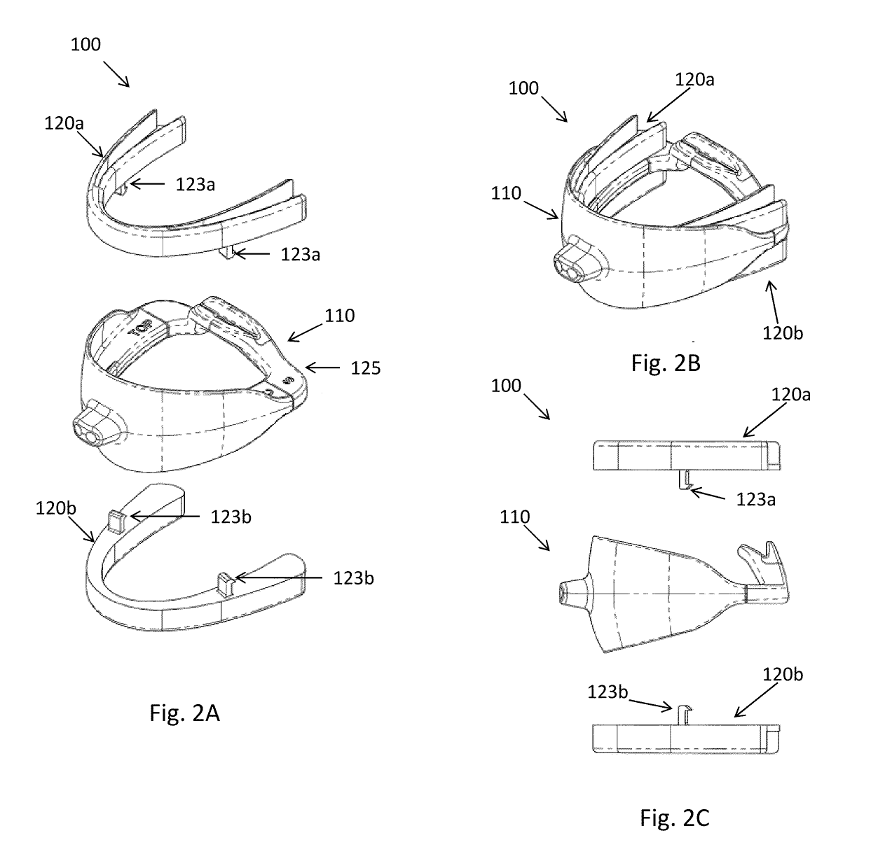

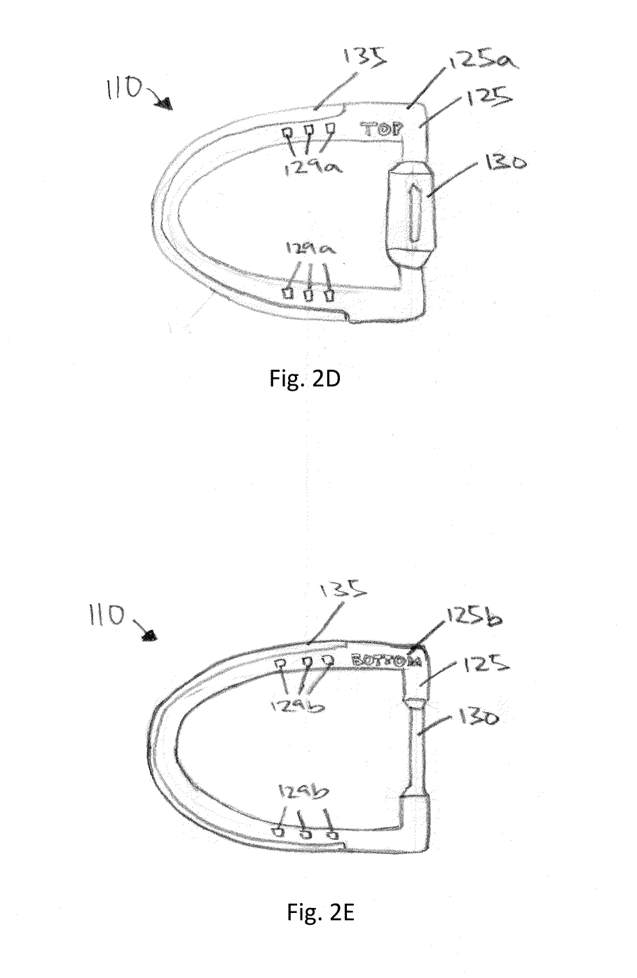

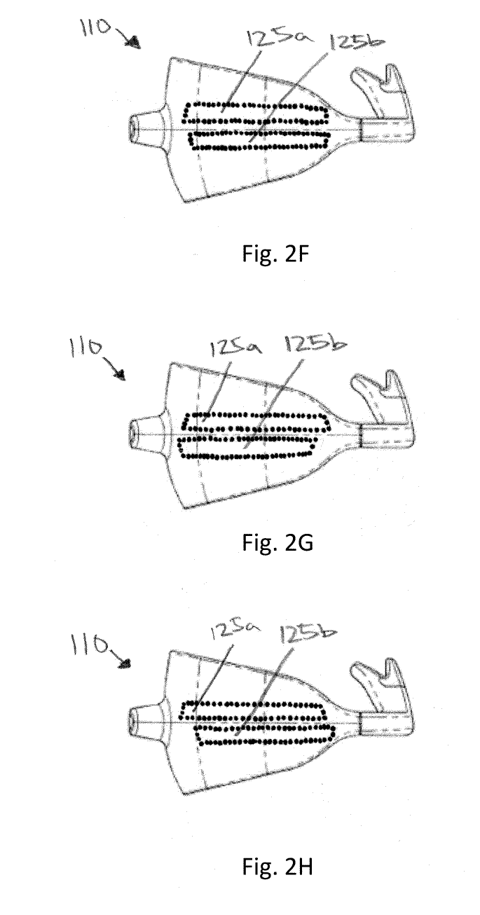

FIG. 2A shows the system 100 which may comprise the treatment device 110 and the upper retainer element 120a and may further comprise a lower retainer element 120b. The lower retainer element 120b may be configured to couple to the patient's lower teeth and may have an interior surface molded to conform to the outer surfaces of the upper teeth of the patient. The upper retainer element 120a may comprise one or more attachment elements 123a or brackets to fasten onto the upper (superior) surface 125a of the U-shaped base 125 of the treatment device 110. The lower retainer element 120b may comprise at least one attachment element 123b or bracket to fasten onto the lower (inferior) surface 125b of the U-shaped base 125 of the treatment device 110. The attachment element(s) 123a, 123b may be separate elements coupled to the upper and lower surfaces 125a, 125b, respectively, of the U-shaped base 125. Alternatively or in combination, the attachment element(s) 123a, 124b may be integral features of the upper and lower surfaces 125a, 125b, respectively, of the U-shaped base 125 which are molded onto the U-shaped base 125. FIG. 2B shows a perspective view upper and lower retainer elements 120a, 120b attached to the treatment device 110. FIG. 2C shows a side view of the treatment device 110 and the upper and lower retainer elements 120a, 120b coupled thereto with the attachment element(s) 123a, 123b

In many embodiments, the upper and lower retainer elements 120a, 120b are positioned relative to one another on the treatment device 110 so that the retainer elements 120a, 120b match the natural bite of the patient when wearing the retainer elements 120a, 120b. Such positioning is shown, for example, in FIG. 2F. Alternatively, the upper and lower retainer elements 120a, 120b may be positioned relative to one another such that the lower teeth are advanced slightly in the anterior direction compared to the natural bite when the retainer elements 120a, 120b are worn. This positioning is shown, for example, in FIG. 2G and can hold the lower teeth and mandible in an advanced position to help maintain airway patency.

As shown in FIGS. 2D and 2E, the treatment device 110 may in some embodiments have a plurality of connection points 129a, 129b for the attachment elements 123a, 123b, respectively. Providing a plurality of connection points 129a, 129b on the top and bottom surfaces 125a, 125b of the U-shaped base 125 can allow the relative positioning of the attachment elements 123a, 123b to be variable and selectable as desired. For example, the upper and lower retainer elements 120a, 120b may be positioned relative to one another such that the retainer elements 120a, 120b match the natural bite of the patient when worn (FIG. 2F), advance the lower teeth and mandible slightly or to varying degrees compared to the natural bite (FIG. 2G), or retract the lower teeth and mandible slightly or to varying degrees compared to the natural bite (FIG. 2H). In a further example, a patient may wear the treatment device 110 such that the retainer elements 120a, 120b matches his/her natural bite on one night and may wear the treatment device 110 such that the retainer elements 120a, 120b advances the lower teeth and mandible on another night.

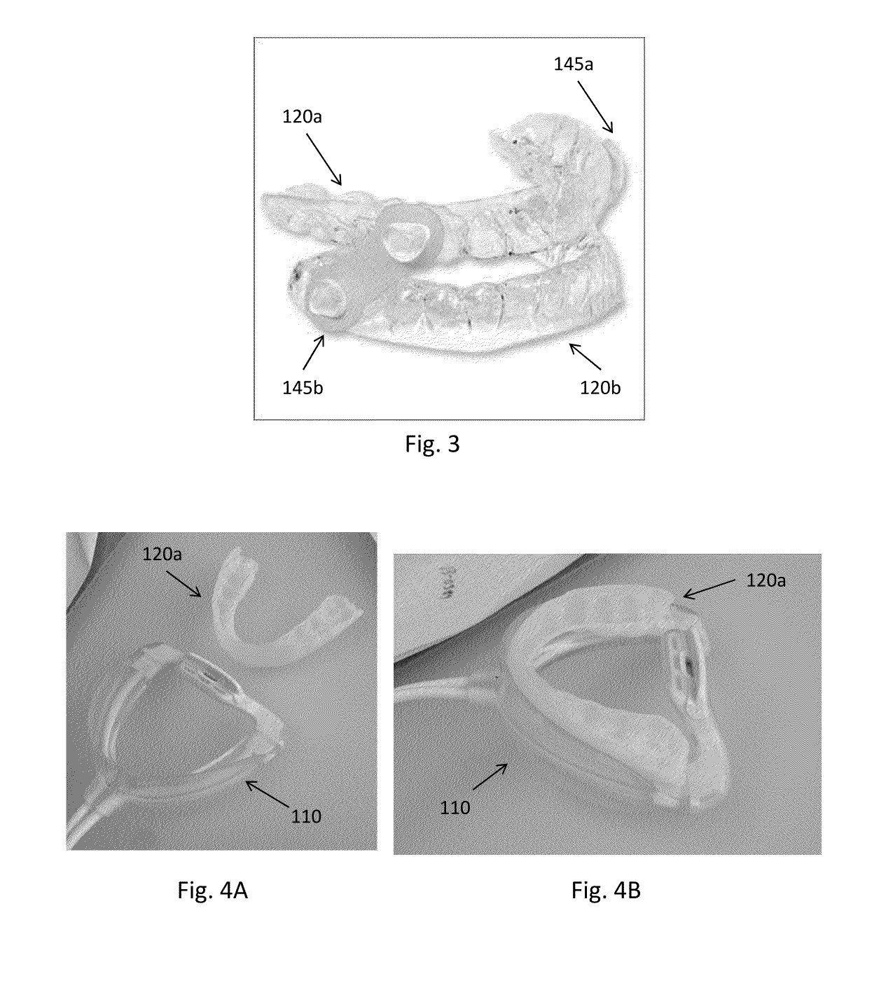

FIG. 3 shows a perspective view of upper and lower retainer elements 120a, 120b coupled together with coupling elements 145a, 145b disposed on each of the two lateral sides of the retainer elements 120a, 120b. The coupling element 145b may be positioned on the upper and lower retainer elements 120a, 120b so that the retainer elements 120a, 120b when worn advance slightly the lower teeth and mandible to help maintain airway patency.

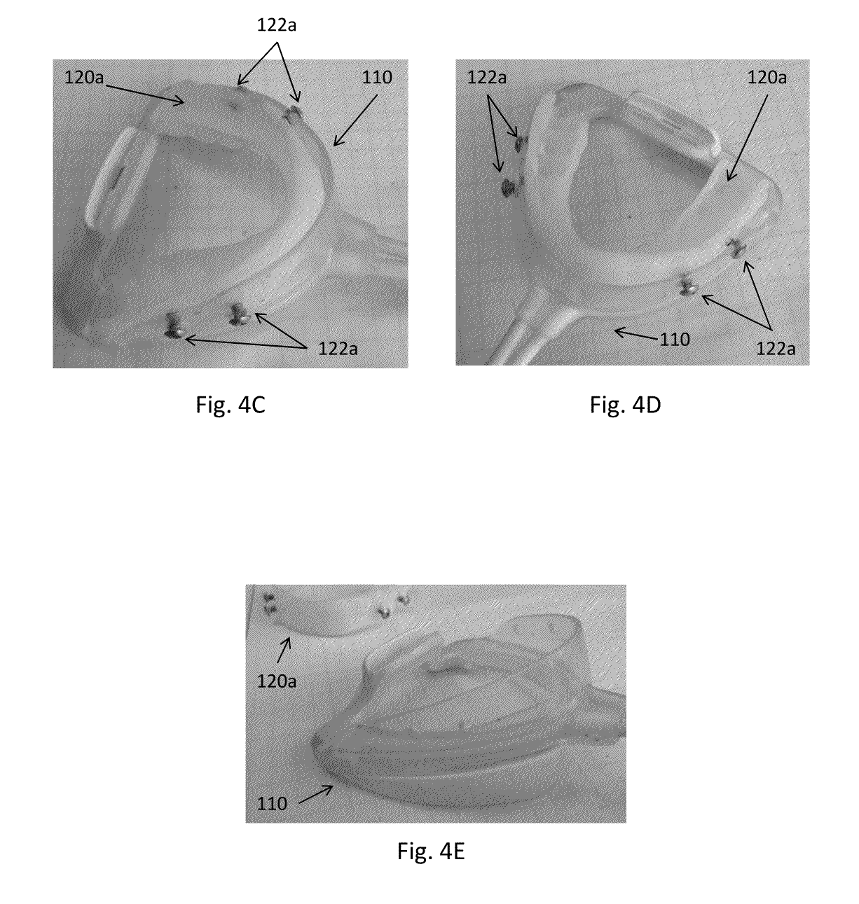

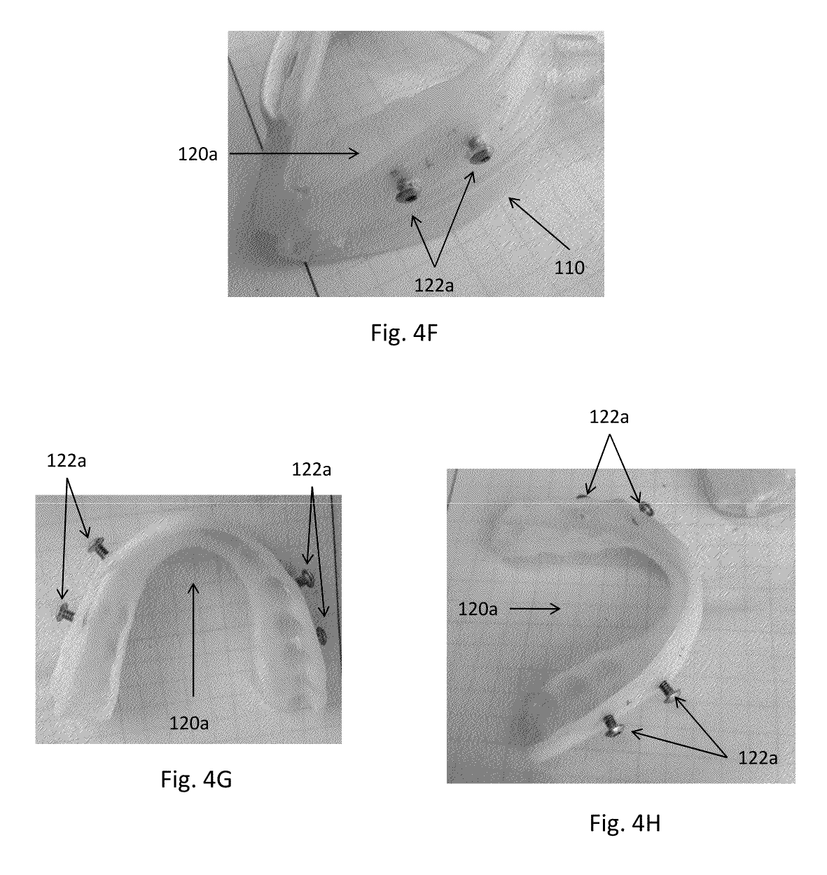

FIG. 4A shows a perspective view of the top of treatment device 110 and the separated upper retainer element 120a. FIG. 4B shows a perspective view of the side of treatment device 110 and the upper retainer element 120a coupled together. FIGS. 4C and 4D show perspective views of the top of treatment device 110 and the upper retainer elements 120a coupled together with the attachment elements 122a. FIG. 4E shows a perspective view of the treatment device 110 placed on a surface. FIG. 4F shows a magnified view of the attachment elements 122a coupling the treatment device 110 and the upper retainer element 120a together. FIG. 4G shows a top view of the upper retainer element 120a coupled to the attachment elements 122a. FIG. 4H shows a perspective view of the top of the upper retainer element 120a coupled to the attachment elements 122a.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An oral device for temporary placement in a patient's oral cavity, said device comprising:
a base adapted to be held between the patient's upper and lower teeth, the base being shaped to accommodate the patient's tongue when held therebetween;
a lip seal for retaining the oral device in the patient's oral cavity and adapted to be held between an inner surface of the patient's lips and an anterior surface of the patient's teeth; and
at least one port adapted to be connected to a vacuum source to apply a partial vacuum in the patient's oral cavity,
wherein at least one of the base or the lip seal is adapted to couple to at least one retainer element, the at least one retainer element being configured to couple and conform to the outer surfaces of at least one of the patient's upper or lower teeth.

2. The oral device of claim 1, further comprising a tongue-engaging member coupled to the base and disposed to engage a medial region of the tongue when the base is positioned between the patient's teeth.

3. The oral device of claim 2, wherein the at least one port is positioned on at least one of the tongue-engaging member or the base.

4. The oral device of claim 3, wherein the at least one port is adapted to be connected to a vacuum source, and wherein the vacuum source is separately connected to and adjustable for each port positioned on the tongue-engaging member and the base.

5. The oral device of claim 1, further comprising a tongue pocket coupled to the base and having an interior disposed to receive an anterior region of the patient's tongue when the tongue passes through an anterior opening of the base.

6. The oral device of claim 5, wherein the tongue pocket extends in an anterior direction from the lip seal.

7. The oral device of claim 5, wherein the at least one port is positioned on at least one of the tongue-engaging member, the base, or the tongue pocket.

8. The oral device of claim 1, further comprising an attachment element coupled to at least one of the base or the lip seal for coupling the at least one retainer element thereto.

9. The oral device of claim 8, wherein the attachment element comprises at least one of a screw, a bolt, a latch, a fastener, or an adhesive.

10. The oral device of claim 1, wherein at least one of the base or the lip seal comprises a first attachment feature configured to couple to a second attachment feature of the at least one retainer element.

11. The oral device of claim 1, wherein the at least one retainer element comprises at least one of an upper retainer element configured to couple and conform to the outer surfaces of the patient's upper teeth or a lower retainer element configured to couple and conform to the outer surfaces of the patient's lower teeth.

12. The oral device of claim 11, wherein the lower retainer element is positioned relative to the upper retainer element on the base such that the patient's lower teeth and mandible are advanced in the anterior direction from their natural position when the patient is wearing the upper and lower retainer elements.

13. The oral device of claim 1, wherein the base comprises upper and lower U-shaped plates each configured to be positioned against full dentition.

14. A system for temporary placement in a patient's oral cavity, said system comprising:
the oral device of claim 1; and
at least one retainer element configured to couple and conform to the outer surfaces of at least one of the patient's upper or lower teeth.

15. The system of claim 14, wherein the at least one retainer element comprises at least one of an upper retainer element configured to couple and conform to the outer surfaces of the patient's upper teeth or a lower retainer element configured to couple and conform to the outer surfaces of the patient's lower teeth.

16. The system of claim 14, further comprising a vacuum source connectable to the oral device.

17. A method for stabilizing a soft palate in a patient's oral cavity, the patient's oral cavity having a tongue and a palate, said method comprising:
placing a bite plate in the oral cavity to be held between upper and lower teeth of the patient;
coupling the bite plate to at least one of the upper or lower teeth of the patient with at least one of an upper or lower retainer element adapted to conform to the outer surfaces of the upper or lower teeth, respectively; and
applying a vacuum to the oral cavity through at least one of the bite plate and a structure coupled to the bite plate.

18. The method of claim 17, wherein applying the vacuum to the oral cavity at least one of draws together the soft palate and a posterior region of the tongue or draws the tongue forward in the anterior direction.

19. The method of claim 17, further comprising drawing the lower teeth and the mandible of the patient forward.

20. The method of claim 19, wherein drawing the lower teeth and the mandible of the patient forward comprises:
positioning the lower retainer element relative to the upper retainer element on the bite plate such that the patient's lower teeth and mandible are advanced in the anterior direction from their natural position; and
coupling the bite plate to the upper and lower teeth of the patient with the upper and lower retainer elements, respectively.

21. The method of claim 17, wherein the structure coupled to the bite plate comprises a tongue-engaging member.

22. The method of claim 21, wherein the vacuum is applied to the oral cavity through at least one ports positioned on at least one of an upper surface of the bite plate, a lower surface of the bite plate, or the tongue-engaging member.

23. The method of claim 21, further comprising engaging the tongue-engaging member against a medial region of the tongue to provide a clearance between the medial region and the palate, wherein the vacuum is applied to the oral cavity though the tongue-engaging member.

24. The method of claim 17, further comprising capturing the tongue in a tongue pocket.

25. The method of claim 24, wherein the vacuum is applied in the pocket to drawn the tongue forward in the anterior direction.

26. An oral device for temporary placement in a patient's oral cavity, said device comprising:
a base adapted to be held between the patient's upper and lower teeth, the base being shaped to accommodate the patient's tongue when held therebetween; and
at least one port adapted to be connected to a vacuum source to apply a partial vacuum in the patient's oral cavity,
wherein the base is adapted to couple to at least one retainer element, the at least one retainer element being configured to couple and conform to the outer surfaces of at least one of the patient's upper or lower teeth.
